SEQUENCE LISTING (11 sequences)

SEQ ID NO: 1 - protein, 477 aa, Mus musculus
MSAAPGLLRQELSCPLCLQLFDAPVTAECGHSFCRACLIRVAGEPAADGTVACPCCQAPTRPQALSTNLQLSRLVEGLAQVPQGHCEEHLDPLSIYCEQDRTLVCGVCASLGSHRGHRLLPAAEAQARLKTQLPQQKMQLQEACMRKEKTVAVLEHQLVEVEETVRQFRGAVGEQLGKMRMFLAALESSLDREAERVRGDAGVALRRELSSLNSYLEQLRQMEKVLEEVADKPQTEFLMKFCLVTSRLQKILSESPPPARLDIQLPVISDDFKFQVWKKMFRALMPALEELTFDPSSAHPSLVVSSSGRRVECSDQKAPPAGEDTRQFDKAVAVVAQQLLSQGEHYWEVEVGDKPRWALGVMAADASRRGRLHAVPSQGLWLLGLRDGKILEAHVEAKEPRALRTPERPPARIGLYLSFADGVLAFYDASNPDVLTPIFSFHERLPGPVYPIFDVCWHDKGKNAQPLLLVGPEQEQA

SEQ ID NO: 2 - protein, 477 aa, Homo sapiens
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 3 - protein, 43 aa, synthetic construct
  REGION 1..43: note = human TRIM72 Ring-finger domain 14-56 aa
CPLCLQLFDAPVTAECGHSFCRACLGRVAGEPAADGTVLCPCC

SEQ ID NO: 4 - protein, 32 aa, synthetic construct
  REGION 1..32: note = human TRIM72 B-box domain 86-117aa)
CEEHLDPLSIYCEQDRALVCGVCASLGSHRGH

SEQ ID NO: 5 - protein, 98 aa, synthetic construct
  REGION 1..98: note = human TRIM72 coiled-coil domain (135-232 aa)
QQKLQLQEACMRKEKSVAVLEHQLVEVEETVRQFRGAVGEQLGKMRVFLAALEGSLDREAERVRGEAGVALRRELGSLNSYLEQLRQMEKVLEEVADK

SEQ ID NO: 6 - protein, 193 aa, synthetic construct
  REGION 1..193: note = human TRIM72 PRYSPRY domain (278-470 aa)
RKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLV

SEQ ID NO: 7 - protein, 421 aa, synthetic construct
  REGION 1..421: note = TRIM72 Ring domain (deletion of 14-69aa)
MSAAPGLLHQELSQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 8 - protein, 435 aa, synthetic construct
  REGION 1..435: note = TRIM72 B-box domain (deletion of 81-122aa)
MSAAPGLLHQELSCPLCLQLFDAPVTAECGHSFCRACLGRVAGEPAADGTVLCPCCQAPTRPQALSTNLQLARLVEGLAQAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 9 - protein, 379 aa, synthetic construct
  REGION 1..379: note = TRIM72 coiled-coil domain (deletion of 135-232aa)
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 10 - protein, 284 aa, synthetic construct
  REGION 1..284: note = TRIM72 PRYSPRY domain (deletion of 278-470aa)
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWGPEGAEA

SEQ ID NO: 11 - protein, 336 aa, synthetic construct
  REGION 1..336: note = TRIM72 coiled-coil domain plus PRYSPRY domain (135-470aa)
QQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLV

(19) United States
(12) Patent Application Publication
(10) Pub. No.: US 2025/0381305 A1
(43) Pub. Date: Dec. 18, 2025

(54) METHOD FOR TREATING A PARKINSON'S DISEASE

(71) Applicant: SINEUGENE THERAPEUTICS CO., LTD., Beijing (CN)

(72) Inventors: Yichang JIA, Beijing (CN); Wei GUO, Beijing (CN); Lin PENG, Beijing (CN)

(21) Appl. No.: 18/879,117

(22) PCT Filed: Jun. 27, 2023
(86) PCT No.: PCT/CN2023/102713

§ 371 Date: Dec. 26, 2024

(30) Foreign Application Priority Data
Jun. 28, 2022 (WO) PCT/CN2022/101917

(51) Int. Cl. A61K 48/00; A61K 38/00; A61P 25/16; C12N 9/10; C12N 15/86

(52) U.S. Cl. CPC A61K 48/0058; A61P 25/16; C12N 9/104; C12N 15/86; A61K 38/00; C07K 2319/00; C12N 2750/14143; C12Y 203/02

(57) ABSTRACT

Provided is a method for treating a Parkinson Disease, comprising a TRIM72 protein modulator. Further provided is a composition comprising the TRIM72 protein modulator and use thereof.

### BACKGROUND OF THE INVENTION

Parkinson's Disease is a disturbance of voluntary movement in which muscles become stiff and sluggish, movement becomes clumsy and difficult and uncontrollable rhythmic twitching of groups of muscles produces characteristic shaking or tremor. The condition is believed to be caused by a degeneration of pre-synaptic dopaminergic neurones in the brain. The absence of adequate release of the chemical transmitter dopamine during neuronal activity thereby leads to the Parkinsonian symptomatology. At present, clinical therapy for PD is still in the stage of “symptomatic treatment”, that is, drugs are selected for the treatment of PD patients with motor or non-motor symptoms. There is a lack of drugs developed based on pathogenesis or pathophysiology of the disease. Drugs for the treatment of motor system disorders mainly target the dopaminergic system pharmacologically, including levodopa, non-ergodopa agonists and monoamine oxidase inhibitors. Levodopa drugs have a significant effect on improving motor symptoms with fast onset and fewer side effects. However, subjects suffer dyskinesia in the later stage, and the dosage and frequency of such drugs are also high. Moreover, treatment of dopamine agonists is likely to cause impulse control disorders and other behavior disorders, or aggravate the patients' psychotic symptoms (such as anxiety, insomnia, etc.). There are few drugs that can target the non-motor symptoms of Parkinson's disease. Although a variety of psychotropic drugs may effectively improve some symptoms of PD patients, whether the use of these drugs will affect PD progression and the safety has yet to be verified. Therefore, there is an urgent need for disease-modifying therapies for PD. Oxidative stress is a critical pathological mechanism of PD, but drugs of antioxidative stress have not been successful so far. Endogenous antioxidant molecular targets are potential breakthroughs in drug development.

Therefore, alternative safe and effective ways for treating Parkinson's disease still need to be explored.

### SUMMARY OF THE INVENTION

The present disclosure provides a method for preventing and/or treating Parkinson's disease. The method can prevent and/or treating the Parkinson's disease safely and effectively.

In one aspect, the present application provides a method for preventing and/or treating Parkinson's disease, comprising administering one or more TRIM72 protein modulators.

In some embodiments, the TRIM72 protein modulator increases the expression and/or activity of said TRIM72 protein.

In some embodiments, the TRIM72 modulator is selected one or more for the group consisting of: a protein, a peptide, a peptidomimetic, a chemical compound, an antibody, a ribozyme, a small molecule chemical compound, a nucleic acid, a vector, and an antisense nucleic acid.

In some embodiments, the TRIM72 protein comprises a TRIM 72 protein or its variant or functional fragment thereof.

In some embodiments, the TRIM72 protein or its functional fragment comprises a human TRIM72 protein or its functional fragment.

In some embodiments, the TRIM72 protein comprises a full-length TRIM72 protein. In some embodiments, the TRIM72 protein comprises a wild type TRIM72 protein.

In some embodiments, the human TRIM72 protein comprises an amino acid sequence as set forth in SEQ ID NO: 2.

In some embodiments, the TRIM72 protein or its functional fragment comprises a TRIM72 truncated protein or its functional fragment.

In some embodiments, the TRIM72 truncated protein comprises the PRYSPRY domain or its functional fragment of a TRIM72 protein.

In some embodiments, the PRYSPRY domain comprises amino acid sites of 278aa-470aa of the TRIM72 protein.

In some embodiments, the PRYSPRY domain comprises an amino acid sequence as set forth in SEQ ID NO: 6.

In some embodiments, the TRIM72 truncated protein further comprises the coiled-coil domain or its functional fragment of a TRIM72 protein.

In some embodiments, the TRIM72 truncated protein does not comprise the coiled-coil domain or its functional fragment of a TRIM72 protein.

In some embodiments, the coiled-coil domain comprises amino acid sites of 135aa-232aa of the TRIM72 protein.

In some embodiments, the coiled-coil domain comprises an amino acid sequence as set forth in SEQ ID NO: 5.

In some embodiments, the TRIM72 truncated protein further comprises the B-box domain or its functional fragment of a TRIM 72 protein.

In some embodiments, the TRIM72 truncated protein does not comprise the B-box domain or its functional fragment of a TRIM72 protein.

In some embodiments, the B-box domain comprises amino acid sites of 86aa-117aa of the TRIM72 protein.

In some embodiments, the B-box domain comprises an amino acid sequence as set forth in SEQ ID NO: 4.

In some embodiments, the TRIM72 truncated protein further comprises the Ring-finger domain or its functional fragment of a TRIM 72 protein.

In some embodiments, the TRIM72 truncated protein does not comprise the Ring-finger domain or its functional fragment of a TRIM72 protein.

In some embodiments, the Ring-finger domain comprises amino acid sites of 14aa-56aa of the TRIM72 protein.

In some embodiments, the Ring-finger domain comprises an amino acid sequence as set forth in SEQ ID NO: 3.

In some embodiments, the TRIM72 truncated protein comprises an amino acid sequence as set forth in any one of SEQ ID NO: 6, 7, 8, 9 and 11.

In some embodiments, the TRIM72 protein or its variant or functional fragment thereof comprises an amino acid mutation at position C14.

In some embodiments, the TRIM72 protein or its variant or functional fragment thereof comprises an amino acid mutation C14A.

In some embodiments, the TRIM72 protein or its variant or functional fragment thereof does not comprise an amino acid mutation at position C242.

In some embodiments, the TRIM72 protein or its variant or functional fragment thereof is secreted through exosome.

In some embodiments, the TRIM72 protein modulator comprises a nucleic molecule encoding said TRIM72 protein or its variant or functional fragment thereof.

In some embodiments, the TRIM72 protein modulator comprises a vector comprising a gene encoding said TRIM72 protein or its variant or functional fragment thereof.

In some embodiments, the vector is a plasmid or virus.

In some embodiments, the vector is an adeno-associated virus (rAAV) expression vector.

In some embodiments, the general promoter is selected one or more from the group consisting of: a chicken β-actin (CBA), a cytomegalovirus (CMV), a CMV immediate enhancer/β-actin (CAG), a truncated CBA hybrid (CBh), an Ubiquitin C (UBC), an elongation factor 1α(EF1A), a mouse or human phosphoglycerate kinase (PGK), a murine stem cell virus (MSCV), spleen focus-forming virus (SFFV), and a simian virus 40 (SV40) promoter.

In some embodiments, the vector comprises a neuron-specific promoter.

In some embodiments, the promoter comprises a human derived promoter.

In some embodiments, the promoter is selected one or more from the group consisting of: an excitatory neuron-specific promoter, a brain neocortical and hippocampal excitatory neuron-specific promoter, a short neuron-specific promoter, a Dopaminergic neuron-specific promoter, a Glutaminergic neuron-specific promoter, a GABAergic neuron-specific promoter, a Cholinergic neuron-specific promoter, and a Serotoninergic neuron-specific promoter.

In some embodiments, the promoter is selected from a group selected from: human synapsin (hSyn), Calcium/calmodulin-dependent kinase IIa (CamKIIa), c-fos, methyl CpG-binding protein 2 (Mecp2), Neuron-specific enolase (NSE), somatostatin (SST), human vesicular GABA (Gamma-Aminobutyric Acid) transporter (hVGAT), choline acetyltransferase (ChAT), Serotonin transporter (SERT) and tyrosine hydroxylase (TH).

In some embodiments, the serotype of said AAV vector is selected from AAV1, AAV2, AAV5, AAV6, AAV8, AAV9, AAVrh, AAVDJ, and AA Vhull.

In some embodiments, the TRIM72 protein modulator comprises a cell, wherein said cell comprises said vector.

In some embodiments, the TRIM72 protein modulator comprises a fusion protein, wherein said fusion protein comprises said TRIM72 protein or its variant or functional fragment thereof.

In some embodiments, the TRIM72 protein modulator prevent and/or treat the Parkinson's disease by reducing oxidative stress to protect neurons.

In another aspect, the present application provides a composition for preventing and/or treating Parkinson's disease, comprising one or more TRIM72 protein modulator.

In some embodiments, the TRIM72 protein modulator increases the expression and/or activity of said TRIM72 protein.

In some embodiments, the TRIM72 modulator is selected one or more for the group consisting of: a protein, a peptide, a peptidomimetic, a chemical compound, an antibody, a ribozyme, a small molecule chemical compound, a nucleic acid, a vector, and an antisense nucleic acid.

In some embodiments, the TRIM72 protein comprises a TRIM 72 protein or its variant or functional fragment thereof.

In some embodiments, the TRIM72 protein or its functional fragment comprises a human TRIM72 protein or its functional fragment.

In some embodiments, the TRIM72 protein comprises a full-length TRIM72 protein.

In some embodiments, the TRIM72 protein comprises a wild type TRIM72 protein.

In some embodiments, the human TRIM72 protein comprises an amino acid sequence as set forth in SEQ ID NO: 2.

In some embodiments, the TRIM72 protein or its functional fragment comprises a TRIM72 truncated protein or its functional fragment.

In some embodiments, the TRIM72 truncated protein comprises the PRYSPRY domain or its functional fragment of a TRIM72 protein.

In some embodiments, the PRYSPRY domain comprises amino acid sites of 278aa-470aa of the TRIM72 protein.

In some embodiments, the PRYSPRY domain comprises an amino acid sequence as set forth in SEQ ID NO: 6.

In some embodiments, the TRIM72 truncated protein further comprises the coiled-coil domain or its functional fragment of a TRIM72 protein.

In some embodiments, the TRIM72 truncated protein does not comprise the coiled-coil domain or its functional fragment of a TRIM72 protein.

In some embodiments, the coiled-coil domain comprises amino acid sites of 135aa-232aa of the TRIM72 protein.

In some embodiments, the coiled-coil domain comprises an amino acid sequence as set forth in SEQ ID NO: 5.

In some embodiments, the TRIM72 truncated protein further comprises the B-box domain or its functional fragment of a TRIM 72 protein.

In some embodiments, the TRIM72 truncated protein does not comprise the B-box domain or its functional fragment of a TRIM72 protein.

In some embodiments, the B-box domain comprises amino acid sites of 86aa-117aa of the TRIM72 protein.

In some embodiments, the B-box domain comprises an amino acid sequence as set forth in SEQ ID NO: 4.

In some embodiments, the TRIM72 truncated protein further comprises the Ring-finger domain or its functional fragment of a TRIM 72 protein.

In some embodiments, the TRIM72 truncated protein does not comprise the Ring-finger domain or its functional fragment of a TRIM72 protein.

In some embodiments, the Ring-finger domain comprises amino acid sites of 14aa-56aa of the TRIM72 protein.

In some embodiments, the Ring-finger domain comprises an amino acid sequence as set forth in SEQ ID NO: 3.

In some embodiments, the TRIM72 truncated protein comprises an amino acid sequence as set forth in any one of SEQ ID NO: 6, 7, 8, 9 and 11.

In some embodiments, the TRIM72 protein or its variant or functional fragment thereof comprises an amino acid mutation at position C14.

In some embodiments, the TRIM72 protein or its variant or functional fragment thereof comprises an amino acid mutation C14A.

In some embodiments, the TRIM72 protein or its variant or functional fragment thereof does not comprise an amino acid mutation at position C242.

In some embodiments, the TRIM72 protein or its variant or functional fragment thereof is secreted through exosome

In some embodiments, the TRIM72 protein modulator comprises a nucleic molecule encoding said TRIM72 protein or its variant or functional fragment thereof.

In some embodiments, the TRIM72 protein modulator comprises a vector comprising a gene encoding said TRIM72 protein or its variant or functional fragment thereof.

In some embodiments, the vector is a plasmid or virus.

In some embodiments, the vector is an adeno-associated virus (rAAV) expression vector.

In some embodiments, the general promoter is selected one or more from the group consisting of: a chicken β-actin (CBA), a cytomegalovirus (CMV), a CMV immediate enhancer/β-actin (CAG), a truncated CBA hybrid (CBh), an Ubiquitin C (UBC), an elongation factor 1α(EF1A), a mouse or human phosphoglycerate kinase (PGK), a murine stem cell virus (MSCV), spleen focus-forming virus (SFFV), and a simian virus 40 (SV40) promoter.

In some embodiments, the vector comprises a neuron-specific promoter.

In some embodiments, the promoter comprises a human derived promoter.

In some embodiments, the promoter is selected one or more from the group consisting of: an excitatory neuron-specific promoter, a brain neocortical and hippocampal excitatory neuron-specific promoter, a short neuron-specific promoter, a Dopaminergic neuron-specific promoter, a Glutaminergic neuron-specific promoter, a GABAergic neuron-specific promoter, a Cholinergic neuron-specific promoter, and a Serotoninergic neuron-specific promoter.

In some embodiments, the promoter is selected from a group selected from: human synapsin (hSyn), Calcium/calmodulin-dependent kinase IIa (CamKIIa), c-fos, methyl CpG-binding protein 2 (Mecp2), Neuron-specific enolase (NSE), somatostatin (SST), human vesicular GABA (Gamma-Aminobutyric Acid) transporter (hVGAT), choline acetyltransferase (ChAT), Serotonin transporter (SERT) and tyrosine hydroxylase (TH).

In some embodiments, the serotype of said AAV vector is selected from AAV1, AAV2, AAV5, AAV6, AAV8, AAV9, AAVrh, AAVDJ, and AA Vhull.

In some embodiments, the TRIM72 protein modulator comprises a cell, wherein said cell comprises said vector.

In some embodiments, the TRIM72 protein modulator comprises a fusion protein, wherein said fusion protein comprises said TRIM72 protein or its variant or functional fragment thereof.

In some embodiments, the TRIM72 protein modulator prevent and/or treat the Parkinson's disease by reducing oxidative stress to protect neurons.

In another aspect, the present application provides a use of TRIM72 protein modulator in manufacture of a medicament for preventing and/or treating Parkinson's disease.

In some embodiments, the TRIM72 protein modulator increases the expression and/or activity of said TRIM72 protein.

In some embodiments, the TRIM72 modulator is selected one or more for the group consisting of: a protein, a peptide, a peptidomimetic, a chemical compound, an antibody, a ribozyme, a small molecule chemical compound, a nucleic acid, a vector, and an antisense nucleic acid.

In some embodiments, the TRIM72 protein comprises a TRIM 72 protein or its variant or functional fragment thereof.

In some embodiments, the TRIM72 protein or its functional fragment comprises a human TRIM72 protein or its functional fragment.

In some embodiments, the TRIM72 protein comprises a full-length TRIM72 protein. In some embodiments, the TRIM72 protein comprises a wild type TRIM72 protein.

In some embodiments, the human TRIM72 protein comprises an amino acid sequence as set forth in SEQ ID NO: 2.

In some embodiments, the TRIM72 protein or its functional fragment comprises a TRIM72 truncated protein or its functional fragment.

In some embodiments, the TRIM72 truncated protein comprises the PRYSPRY domain or its functional fragment of a TRIM72 protein.

In some embodiments, the PRYSPRY domain comprises amino acid sites of 278aa-470aa of the TRIM72 protein.

In some embodiments, the PRYSPRY domain comprises an amino acid sequence as set forth in SEQ ID NO: 6.

In some embodiments, the TRIM72 truncated protein further comprises the coiled-coil domain or its functional fragment of a TRIM72 protein.

In some embodiments, the TRIM72 truncated protein does not comprise the coiled-coil domain or its functional fragment of a TRIM72 protein.

In some embodiments, the coiled-coil domain comprises amino acid sites of 135aa-232aa of the TRIM72 protein.

In some embodiments, the coiled-coil domain comprises an amino acid sequence as set forth in SEQ ID NO: 5.

In some embodiments, the TRIM72 truncated protein further comprises the B-box domain or its functional fragment of a TRIM 72 protein.

In some embodiments, the TRIM72 truncated protein does not comprise the B-box domain or its functional fragment of a TRIM72 protein.

In some embodiments, the B-box domain comprises amino acid sites of 86aa-117aa of the TRIM72 protein.

In some embodiments, the B-box domain comprises an amino acid sequence as set forth in SEQ ID NO: 4.

In some embodiments, the TRIM72 truncated protein further comprises the Ring-finger domain or its functional fragment of a TRIM 72 protein.

In some embodiments, the TRIM72 truncated protein does not comprise the Ring-finger domain or its functional fragment of a TRIM72 protein.

In some embodiments, the Ring-finger domain comprises amino acid sites of 14aa-56aa of the TRIM72 protein.

In some embodiments, the Ring-finger domain comprises an amino acid sequence as set forth in SEQ ID NO: 3.

In some embodiments, the TRIM72 truncated protein comprises an amino acid sequence as set forth in any one of SEQ ID NO: 6, 7, 8, 9 and 11.

In some embodiments, the TRIM72 protein or its variant or functional fragment thereof comprises an amino acid mutation at position C14.

In some embodiments, the TRIM72 protein or its variant or functional fragment thereof comprises an amino acid mutation C14A.

In some embodiments, the TRIM72 protein or its variant or functional fragment thereof does not comprise an amino acid mutation at position C242.

In some embodiments, the TRIM72 protein or its variant or functional fragment thereof is secreted through exosome.

In some embodiments, the TRIM72 protein modulator comprises a nucleic molecule encoding said TRIM72 protein or its variant or functional fragment thereof.

In some embodiments, the TRIM72 protein modulator comprises a vector comprising a gene encoding said TRIM72 protein or its variant or functional fragment thereof.

In some embodiments, the vector is a plasmid or virus.

In some embodiments, the vector is an adeno-associated virus (rAAV) expression vector.

In some embodiments, the general promoter is selected one or more from the group consisting of: a chicken β-actin (CBA), a cytomegalovirus (CMV), a CMV immediate enhancer/β-actin (CAG), a truncated CBA hybrid (CBh), an Ubiquitin C (UBC), an elongation factor 1α(EF1A), a mouse or human phosphoglycerate kinase (PGK), a murine stem cell virus (MSCV), spleen focus-forming virus (SFFV), and a simian virus 40 (SV40) promoter.

In some embodiments, the vector comprises a neuron-specific promoter.

In some embodiments, the promoter comprises a human derived promoter.

In some embodiments, the promoter is selected one or more from the group consisting of: an excitatory neuron-specific promoter, a brain neocortical and hippocampal excitatory neuron-specific promoter, a short neuron-specific promoter, a Dopaminergic neuron-specific promoter, a Glutaminergic neuron-specific promoter, a GABAergic neuron-specific promoter, a Cholinergic neuron-specific promoter, and a Serotoninergic neuron-specific promoter.

In some embodiments, the promoter is selected from a group selected from: human synapsin (hSyn), Calcium/calmodulin-dependent kinase IIa (CamKIIa), c-fos, methyl CpG-binding protein 2 (Mecp2), Neuron-specific enolase (NSE), somatostatin (SST), human vesicular GABA (Gamma-Aminobutyric Acid) transporter (hVGAT), choline acetyltransferase (ChAT), Serotonin transporter (SERT) and tyrosine hydroxylase (TH).

In some embodiments, the serotype of said AAV vector is selected from AAV1, AAV2, AAV5, AAV6, AAV8, AAV9, AAVrh, AAVDJ, and AA Vhull.

In some embodiments, the TRIM72 protein modulator comprises a cell, wherein said cell comprises said vector.

In some embodiments, the TRIM72 protein modulator comprises a fusion protein, wherein said fusion protein comprises said TRIM72 protein or its variant or functional fragment thereof.

In some embodiments, the TRIM72 protein modulator prevent and/or treat the Parkinson's disease by reducing oxidative stress to protect neurons.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates different TRIM72 constructs and TRIM40 construct in pAAV-ITR vectors.

FIG. 2 illustrates schematic diagram showing the timeline of treatments and behavior tests in mice underwent MPTP modeling and scAAV-TRIM72 treatment.

FIG. 3 illustrates performance of rotarod behavior. The stay time on the rotarod was recorded in the experiments. (A) MPTP administration shortened the residence time. (B) The mice injected with scAAVs-TRIM72 had a significantly longer residence time. The values are presented as mean±SEM with all data points. Student t-test or one-way ANOVA was performed to compare the datasets, ***p<0.001 (t-test), *p<0.05 (ANOVA), N.S., no statistical significance.

FIG. 4 illustrates the number of TH-positive dopaminergic (DA) neurons in the substantia nigra region. (A) MPTP administration resulted in a significant reduction of DA neurons, (B) whereas scAAV-TRIM72 injection effectively prevented the loss of these motor neurons. The values are presented as mean±SEM with all data points. Student t-test was performed to compare the datasets, ***p<0.001, *p<0.05, N.S., no statistical significance.

FIG. 5 illustrates the expression pattern of TRIM72 in exosomes by western blot analysis.

FIG. 6 illustrates the secretion efficiency of full-length or different-truncated TRIM72 in exosomes. The values are presented as mean±SEM with all data points. One-way ANOVA was performed to compare the datasets, ***p<0.001, N.S., no statistical significance.

FIG. 7 illustrates TRIM72 domain annotation and the key residues for TRIM72 functions.

FIG. 8 illustrates expression of domain-deleted TRIM72 fused with Flag tag and EGFP. GAPDH served as protein loading control. FL, full-length TRIM72.

FIG. 9 illustrates the effect of domain-deleted TRIM72 on cell viability after stress challenge. The Coiled-coil domain- or PRYSPRY deleted not Ring domain- or B-box deleted abolished the protective effect of TRIM72. Values are presented as mean±SEM and student t-test was performed to compare the datasets, ***p<0.001, **p<0.01, N.S., no statistical significance.

FIG. 10 illustrates the cell viability under H₂O₂ stress with expression of full-length TRIM72, various TRIM72 truncation, or full-length TRIM40 by scAAV9 infection. The values are presented as mean±SEM with all data points. Student t-test was performed to compare the datasets with control, **p<0.01, *p<0.05, N.S., no statistical significance.

FIG. 11 illustrates the expression of the TRIM72 mutants on cell viability after stress challenge. The C242A but not C14A mutation abolished the anti-stress effect of TRIM72. The values were generated from at least three independent experiments (n>3). One-way ANOVA was performed to compare the datasets, *p<0.05, **p<0.01, ***p<0.001. N.S., no statistical significance.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In the present application, the term “TRIM72 protein modulator” generally refers to a substance or means that modifies the expression, activity and/or biological function of TRIM72 protein as compared to the expression, activity and/or biological function of the TRIM72 protein in the absence of the modulator. The modulator can include but not limited to a chemical compound, a protein, a peptide, a peptidomemetic, an antibody, a ribozyme, a small molecule chemical compound, a nucleic acid, a vector, and an antisense nucleic acid.

In the present application, the term “Parkinson's disease” generally refers to a kind of neurodegenerative disorder. The Parkinson's disease is a chronic progressive nervous disease characterised by neurodegeneration, especially degeneration of dopaminergic neurons. Symptoms include stooped posture, resting tremor, weakness of resting muscles, a shuffling gait, speech impediments, movement difficulties and an eventual slowing of mental processes and/or dementia.

In the present application, the term “TRIM72 protein” can be used interchangeably with “MG53” protein, generally include a TRIM72 protein or its variant, functional fragment, analogue, homologue. The TRIM72 protein may contain a Ring finger, a B-box motif, a coiled-coil region and/or a C-terminal PRYSPRY domain. For example, the Ring-finger domain may comprise amino acid sites of 14aa-56aa of the TRIM72 protein or its functional fragment. For example, the B-box domain may comprise amino acid sites of 86aa-117aa of the TRIM72 protein or its functional fragment. For example, the coiled-coil domain may comprise amino acid sites of 135aa-232aa of the TRIM72 protein or its functional fragment. For example, the PRYSPRY domain may comprise amino acid sites of 278aa-470aa of the TRIM72 protein. The term may also include the TRIM72 protein derived from any known species which has a TRIM72 protein.

In the present application, the term “truncated protein” generally refers to a protein with one or more amino acid deletion compared with the full-length protein. For example, the truncated protein may contain the main functional fragment of the protein. For example, the truncated protein also includes but not limited to its variant, functional fragment, analogue, homologue.

In the present application, the “amino acid mutation Xn” refers to an amino acid mutation occurring in the amino acid residue X at position n of the amino acid sequence as set forth in SEQ ID NO: 2, wherein n is a positive integer, X is an abbreviation of any amino acid residue. For example, the “amino acid mutation C14” refers to the amino acid substitution occurring in the amino acid residue C corresponding to position 14 of the amino acid sequence as set forth in SEQ ID NO: 2.

The amino acid mutations of the present application can be non-conserved mutations. Said non-conserved mutations can comprise changing the amino acid residues in a target protein or polypeptide in a non-conserved manner, e.g., replacing an amino acid residue having a certain side chain size or a certain characteristic (e.g., hydrophilic) with an amino acid residue having a different side chain size or a different characteristic (e.g., hydrophobic).

Said amino acid substitutions can also be conserved substitutions. Said conserved substitutions can comprise changing the amino acid residues in a target protein or polypeptide in a conserved manner, e.g., replacing an amino acid residue having a certain side chain size or a certain characteristic (e.g., hydrophilic) with an amino acid residue having the same or similar side chain size or the same or similar characteristic (e.g., still hydrophilic). Such conserved substitutions generally would not produce a significant effect on the structure or the function of the produced protein. In the present application, the amino acid sequence variant which is a mutant of the fusion protein, its fragment, or its variant which undergoes one or more amino acid substitutions can comprise conserved amino acid substitutions that would not remarkably change the structure or function of the protein.

As an example, the mutual substitutions between amino acids in each of the following groups can be considered as conservative substitutions in the present application:
Group of amino acids with nonpolar side side(s): alanine, valine, leucine, isoleucine, proline, phenylalanine, tryptophan and methionine.
Group of uncharged amino acids with polar side chains: glycine, serine, threonine, cysteine, tyrosine, asparagine and glutamine.
Group of negatively charged amino acids with polar side chains: aspartic acid and glutamic acid.
Group of positively charged basic amino acids: lysine, arginine and histidine.
Group of amino acids with phenyl: phenylalanine, tryptophan and tyrosine.

In the present application, the term “fusion protein” generally refers to a complex polypeptide, that is, a single continuous amino acid sequence consisting of two (or more) polypeptides. The fusion protein can generally be artificially prepared by means of recombinant nucleic acid or chemical synthesis.

In the present application, the term “nucleic acid molecule” generally refers to an isolated form of nucleotide, deoxyribonucleotide or ribonucleotide or their analogs of any length isolated from their natural environment or artificially synthesized. The nucleic acid molecules of the present application can be isolated. For example, it can be produced or synthesized by the following ways: (i) in vitro amplification, such as polymerase chain reaction (PCR) amplification, (ii) clonal recombination, (iii) purification, e.g., fractionation by restriction enzyme digestion and gel electrophoresis, or (iv) synthesis, e.g., chemical synthesis. In some embodiments, said isolated nucleic acid is a nucleic acid molecule prepared by a recombinant DNA technology. In the present application, the nucleic acid encoding said truncated protein or its functional fragment can be prepared by a variety of methods known in the art. These methods include, but are not limited to, overlap extension PCR by use of restriction fragment operations or synthetic oligonucleotides. Specific operations can be found in Sambrook et al., Molecular Cloning, A Laboratory Manual, Cold Spring Harbor Laboratory Press, Cold Spring Harbor, NY, 1989; and Ausube et al. Current Protocols in Molecular Biology, Greene Publishing and Wiley-Interscience, New York NY, 1993.

In the present application, the term “vector” generally refers to a vector containing a recombinant polynucleotide, where the recombinant polynucleotide includes an expression control sequence efficiently linked to a nucleotide sequence to be expressed. The vector includes cis-acting elements sufficient for expression; other elements for expression may be provided by the host cell or may be provided in an in-vitro expression system. The vector may include all expression vectors known in the art that can be incorporated into the recombinant polynucleotide, including cosmid, plasmid (e.g., naked or encapsulated in liposomes) and viruses (e.g., lentiviruses, retroviruses, adenoviruses, and adeno-associated viruses).

In the present application, the term “encoding” generally refers to the inherent property of a particular sequence of nucleotides in a polynucleotide such as a gene, cDNA or mRNA to act as a template for the synthesis of other multimers and macromolecules in a biological process, said multimers and macromolecules having either a defined sequence of nucleotides (i.e., rRNA, tRNA and mRNA) or a defined sequence of amino acids and the biological properties arising therefrom. Thus, if transcription and translation of an mRNA corresponding to a gene produces a protein in a cell or other biological system, the gene encodes the protein. Both the coding strand whose nucleotide sequence is identical to the mRNA sequence and is usually provided in the sequence listing, and the non-coding strand used as a template for the transcription of a gene or cDNA may be referred to as the protein or other product encoding the gene or cDNA. In the present application, the term “coding element” generally refers to a nucleic acid (an RNA or DNA molecule) including a nucleotide sequence encoding a protein.

In the present application, the terms “host cell”, “cell”, and “host” are used interchangeably, and generally refer to a plasmid or vector that can include or have included the nucleic acid molecule of the present application, or can express individual cells, cell lines or cell cultures of the protein of the present application, its fragments or its variants. Said host cell can comprise the progeny of a single host cell. Due to natural, accidental or deliberate mutations, the progeny cells and the original parent cells can not necessarily be completely identical in morphology or genome, as long as they can express the protein of the present application or its fragments. Said host cell can be obtained by transfecting cells in vitro with the vector of the present application. Said host cell can be a prokaryotic cell (e.g., Escherichia coli) or a eukaryotic cell (e.g., yeast cells, e.g., COS cells, Chinese Hamster Ovary (CHO) cells, HeLa cells, HEK293 cells, COS-1 cells, NSO cells or myeloma cells). In the present application, said host cell can be a CHO cell.

In the present application, the term “treat” generally refers to slowing or improving the progression, severity, and/or duration of a proliferative condition, or improving one or more symptoms (e.g., one or more distinguishable symptoms) of a proliferative condition as a result of the administration of one or more therapies.

In the present application, the term “subject” generally refers to any human or non-human animal. The term “non-human animal” can include all vertebrates, such as, mammals and non-mammals, e.g., non-human primates, goats, sheep, dogs, cows, chickens, amphibians, reptiles, etc.

In the present application, the terms “peptide”, “polypeptide” and “protein” can be used interchangeably and generally refer to compounds composed of amino acid residues covalently linked by peptide bonds. The protein or peptide must contain at least two amino acids, and there is no limitation on the maximum number of amino acids that can be included in the protein or peptide sequence. The polypeptide may include any peptides or proteins that contain two or more amino acids linked to each other through peptide bonds. In the present application, this term refers to two short chains, which are also commonly known as peptides, oligopeptides and oligomers in the art, for example longs chains, which are commonly known as proteins in the art, of which there are many types. “Polypeptides” include, for example, bioactive fragments, substantially homologous polypeptides, oligopeptides, homodimers, heterodimers, variants of polypeptides, modified polypeptides, derivatives, analogues, fusion proteins, etc. Polypeptides include native peptides, recombinant peptides or combinations thereof.

In addition to particular proteins and nucleotides mentioned herein, the present application may also include their functional variants, derivatives, analogues, homologues and fragments thereof.

The term “functional variant” refers to a polypeptide having substantially the same amino acid sequence or encoded by substantially the same nucleotide sequence as the naturally occurring sequence and capable of having one or more activities of the naturally occurring sequence. In the context of the present application, the variant of any given sequence refers to a sequence in which a particular sequence of residues (either amino acid or nucleotide residues) has been modified so that the polypeptide or polynucleotide remains substantially at least one endogenous function. The variant sequences can be obtained through the addition, deletion, substitution, modification, replacement and/or variation of at least one amino acid residue and/or nucleotide residue present in a naturally occurring protein and/or polynucleotide, as long as the original functional activity is maintained. In the present application, the term “derivative” generally refers to a polypeptide or polynucleotide of the present application including any substitution, variation, modification, replacement, deletion and/or addition from/to one (or more) amino acid residues of the sequence, provided that the resulting polypeptide or polynucleotide substantially maintains at least one of its endogenous functions.

In the present application, the term “analogue” generally, with respect to a polypeptide or polynucleotide, includes any mimetic of the polypeptide or polynucleotide, that is, a chemical compound having at least one endogenous function of the polypeptide or polynucleotide that the mimetic mimics. In general, amino acids can be substituted, for example, at least 1 (e.g., 1, 2, 3, 4, 5, 6, 7, 8, 9, 10 or 20 or above) amino acids can be substituted, provided that the modified sequence substantially maintains the required activity or capability. Amino acid substitution may include the use of non-naturally occurring analogues. The protein or polypeptide used in the present application may also have deletion, insertion or substitution of amino acid residues, where the amino acid residues undergo silent changes and result in functionally equivalent proteins. Intentional amino acid substitutions can be made based on the similarity of the polarity, charge, solubility, hydrophobicity, hydrophilicity, and/or the amphoteric properties of the residues, as long as the endogenous function is retained. For example, negatively charged amino acids include aspartic acid and glutamic acid; positively charged amino acids include lysine and arginine; and amino acids containing uncharged polar head-groups with a similar hydrophilic value include asparagine, glutamine, serine, threonine and tyrosine.

In the present application, the term “homologue” generally refers to an amino acid sequence or a nucleotide sequence having a certain homology with a wild-type amino acid sequence and a wild-type nucleotide sequence. The term “homology” may be equivalent to the “identity” of sequences. Homologous sequences may include amino acid sequences that are at least 80%, 85%, 90%, 99.1%, 99.2%, 99.3%, 99.4%, 99.5%, 99.6%, 99.7%, 99.8% or 99.9% the same as the subject sequence. In general, homologues will contain the same active sites as the subject amino acid sequence, and the like. Homology may be considered on the basis of similarity (i.e., amino acid residues having similar chemical properties/functions), or homology can be expressed in terms of the sequence identity. In the present application, a sequence having a percentage identity in either of the SEQ ID NOs of the mentioned amino acid sequence or nucleotide sequence refers to a sequence having the percentage identity over the whole length of the mentioned SEQ ID NOs. In order to determine the sequence identity, alignment of sequences can be performed by a variety of ways known to those skilled in the art, for example, by using BLAST, BLAST-2, ALIGN, NEEDLE or Megalign (DNASTAR) software, etc. The persons skilled in the art are able to determine the suitable parameters suitable for alignment, including any algorithms required to achieve an optimal alignment in the full-length sequence being compared.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below a specified value.

In the present application, the term “comprising” usually means including, containing, having or encompassing. In some cases, it also refers to the meaning of “being” or “consisting of”.

In the present application, the term “does not comprise” generally refers to the exclusion of the possibility of a certain behavior, structure or structure. For example, “A does not comprise B” generally means to exclude the possibility of B occurring in A.

### Method, Use and Preparation

In one aspect, the present application provides a method for preventing and/or treating Parkinson's disease, comprising administering one or more TRIM72 protein modulators.

In another aspect, the present application provides a composition for preventing and/or treating Parkinson's disease, comprising one or more TRIM72 protein modulator.

In another aspect, the present application provides a use of TRIM72 protein modulator in manufacture of a medicament for preventing and/or treating Parkinson's disease.

In the present application, the TRIM72 protein modulator can increase the expression and/or activity of said TRIM72 protein.

In the present application, the TRIM72 modulator can be selected one or more for the group consisting of: a protein, a peptide, a peptidomimetic, a chemical compound, an antibody, a ribozyme, a small molecule chemical compound, a nucleic acid, a vector, and an antisense nucleic acid.

In the present application, the TRIM72 protein may comprise a TRIM 72 protein or its variant or functional fragment thereof.

In the present application, the TRIM72 protein modulator can comprise a vector. For example, the vector can comprise a recombinant adeno-associated virus (rAAV) expression vector, comprising a gene encoding a TRIM72 protein or its variant or functional fragment thereof.

In the present application, the TRIM72 protein or its functional fragment may comprise a human TRIM72 protein or its functional fragment.

In the present application, the TRIM72 protein can comprise a full-length TRIM72 protein. In the present application, the TRIM72 protein can comprise a wild type TRIM72 protein. In the present application, the human TRIM72 protein comprises an amino acid sequence as set forth in SEQ ID NO: 2.

In the present application, the TRIM72 protein can comprise a TRIM72 truncated protein.

In the present application, the TRIM72 truncated protein comprises the PRYSPRY domain or its functional fragment. For example, the TRIM72 truncated protein may comprises amino acid sites of 278aa-470aa of the TRIM72 protein. For example, the TRIM72 truncated protein may comprise an amino acid sequence as set for in SEQ ID NO: 6.

In the present application, the TRIM72 truncated protein may further comprise other domain of TRIM72 protein.

For example, the TRIM72 truncated protein may comprise the PRYSPRY domain and coiled-coil domain. For example, the TRIM 72 truncated protein may comprise a deletion of B-box domain and Ring-finger domain of TRIM72 protein. For example, the TRIM72 protein may comprise an amino acid sequence as set forth in SEQ ID NO: 11.

For example, the TRIM72 truncated protein may comprise the PRYSPRY domain and B-box domain. For example, the TRIM 72 truncated protein may comprise a deletion of coiled-coil domain and Ring-finger domain of TRIM72 protein.

For example, the TRIM72 truncated protein may comprise the PRYSPRY domain and the Ring-finger domain. For example, the TRIM 72 truncated protein may comprise a deletion of B-box domain and coiled-coil domain of TRIM72 protein.

For example, the TRIM72 truncated protein may comprise the PRYSPRY domain, the coiled-coil domain and the Ring-finger domain. For example, the TRIM 72 truncated protein may comprise a deletion of B-box domain of TRIM72 protein.

For example, the TRIM72 truncated protein may comprise the PRYSPRY domain, the coiled-coil domain and the B-box domain. For example, the TRIM 72 truncated protein may comprise a deletion of Ring-finger domain of TRIM72 protein.

For example, the TRIM72 truncated protein may comprise the PRYSPRY domain, the Ring-finger domain and the B-box domain. For example, the TRIM 72 truncated protein may comprise a deletion of coiled-coil domain of TRIM72 protein.

In the present application, the TRIM72 protein or its fragments may comprise its variants. For example, the TRIM72 protein may comprise one or more amino acid mutations compared with the correspondence wild type sequence.

In the present application, the TRIM72 protein may comprise an amino acid mutation at position C14. For example, the amino acid mutation may be C14A.

According to applicant's research, amino acid cysteine at position 242 is critical for oligomer formation of TRIM72 protein. Substitution of amino acid C242 may block the protection function of neurons. Therefore, amino acid substitution at position C242 (for example, C242A) may not be included in the TRIM72 protein in the present application.

Exosomes are small extracellular biological vesicles released into surrounding body fluids through fusion of multivesicular bodies and the plasma membrane, which contain proteins, nucleic acids, lipids and other bioactive substances. In the present application, the TRIM72 protein or its variant, or its fragments thereof may be secreted through exosome. For example, vectors comprising gene encoding the TRIM72 protein or its variant, or its fragments thereof may be constructed and expressed in host cell. TRIM72 protein or its variant, or its fragments thereof could affect the biological processes of surrounding cells through the exosomal secretion pathway.

In the present application, the rAAV may comprise an AAV genome or a derivative thereof, and/or an AAV capsid protein or a derivative thereof. In the present application, the rAAV may be a chimeric AAV, a shuffled AAV, or a capsid-modified AAV. In the present application, the AAV genome or AAV capsid protein may be from any one of AAV1, AAV2, AAV3, AAV4, AAV5, AAV6, AAV7, AAV8, AAV9, AAV10, AAV11, AAV12, AAVrh, AAVDJ, and AAVhull. In the present application, the rAAV may be a hybrid AAV (e.g., AAV-DJ, AAV-DJ/8, or AAV-DJ/9). In the present application, the rAAV may be developed through directed evolution and/or rational design (e.g., AAV 7m8 or AAV-PHP.eB).

In the present application, the rAAV expression vector may comprise a general promoter. In the present application, the general promoter may be selected from a chicken β-actin (CBA), a cytomegalovirus (CMV), a CMV immediate enhancer/β-actin (CAG), a truncated CBA hybrid (CBh), an Ubiquitin C (UBC), an elongation factor 1α(EF1A), a mouse or human phosphoglycerate kinase (PGK), a murine stem cell virus (MSCV), spleen focus-forming virus (SFFV), or a simian virus 40 (SV40) promoter.

In the present application, the rAAV expression vector may comprise a neuron-specific promoter. For example, the promoter can comprise a human derived promoter.

In the present application, the promoter may be selected one or more from the group consisting of: an excitatory neuron-specific promoter, a brain neocortical and hippocampal excitatory neuron-specific promoter, a short neuron-specific promoter, a Dopaminergic neuron-specific promoter, a Glutaminergic neuron-specific promoter, a GABAergic neuron-specific promoter, a Cholinergic neuron-specific promoter, and a Serotoninergic neuron-specific promoter.

In some embodiments, the promoter may be selected from a group selected from: human synapsin (hSyn), Calcium/calmodulin-dependent kinase IIa (CamKIIa), c-fos, methyl CpG-binding protein 2 (Mecp2), Neuron-specific enolase (NSE), somatostatin (SST), human vesicular GABA (Gamma-Aminobutyric Acid) transporter (hVGAT), choline acetyltransferase (ChAT), Serotonin transporter (SERT) and tyrosine hydroxylase (TH).

In the present application, the TRIM72 protein may comprise a recombinant protein comprising the TRIM72 protein or its variant or functional fragment thereof.

In the present application, the TRIM72 protein modulator may comprise one or more nucleic acid molecules capable of encoding the TRIM72 protein or its variant or functional fragment thereof.

In the present application, the TRIM72 protein modulator may comprise one or more vectors which can comprise one or more nucleic acid molecules of the present application. In the present application, the TRIM72 protein modulator may comprise a cell (e.g., a host cell), which can comprise the nucleic acid molecule of the present application or the vector of the present application.

In the present application, the vector can be a polynucleotide that can be transcribed and translated into a polypeptide when introduced into a suitable host cell. Generally, by culturing a suitable host cell containing said vector, said vector can produce the desired expression product. In the present application, said vector can include one or more of said nucleic acid molecules. For example, said vector can comprise all the nucleic acid molecules required for encoding said TRIM72 protein or its variant or functional fragment thereof.

In addition, said vector can also include other genes, such as a marker gene that allows selecting the vector in a suitable host cell and under suitable conditions. In addition, said vector can also include an expression control element that allows the coding region to be properly expressed in a suitable host. Such control element is well known to those skilled in the art. For example, they can comprise promoters, ribosome binding sites, enhancers, and other control elements that regulate gene transcription or mRNA translation. In some embodiments, said expression control sequence is a regulatory element. The specific structure of said expression control sequence can vary depending on the function of the species or cell types, but usually comprises 5′ non-transcribed sequences and 5′ and 3′ non-translated sequences involved in transcription and translation initiation, such as TATA boxes, capped sequences, CAAT sequences, etc. For example, the 5′ non-transcribed expression control sequence can comprise a promoter region, and the promoter region can comprise a promoter sequence for transcriptional control of the functionally linked nucleic acid.

In the present application, the composition may comprise one or more TRIM72 modulator.

In the present application, the composition may comprise one or more TRIM72 modulator, and a pharmaceutically accepted adjuvant.

In some embodiments, pharmaceutically acceptable adjuvant can comprise buffers, antioxidants, preservatives, low molecular weight polypeptides, proteins, hydrophilic polymers, amino acids, sugars, chelating agents, counter-ions, metal complexes, and/or nonionic surfactants etc.

In some embodiments, the pharmaceutically accepted adjuvant can comprise drug, toxins, cytokines, radioactive elements, carrier proteins, enzymes, lectins, fluorescent quantum dots, and/or high absorption coefficient of chromophore.

In the present application, said composition can be formulated with a pharmaceutically acceptable carrier or diluent and any other known adjuvants and excipients according to conventional technical means in the art, e.g., following the operations in Remington: The Science and Practice of Pharmacy, nineteenth edition, edited by Gennaro, Mack Publishing Co., Easton, PA, 1995.

In the present application, said composition can be formulated for oral administration, intravenous administration, intramuscular administration, in situ administration at the tumor site, inhalation, rectal administration, vaginal administration, transdermal administration or the medicine is administered via a subcutaneous depot.

In the present application, said composition can be used to protect neurons. For example, the composition of the present application can inhibit or delay the development or progression of Parkinson's disease, and/or can reduce and/or stabilize the disease status.

The composition of the present application can comprise a therapeutically effective amount of said TRIM72 protein modulator. Said therapeutically effective amount is a dose required to prevent and/or treat (at least partially treat) Parkinson's disease and/or any complications thereof in a subject with or at a risk of the diseases.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Methods

### MPTP Modeling

5-week-old C57B16/J male mice were used for MPTP-induced PD modeling. The mice were injected scAAV through retro-orbital intravenous injection at 14 days before the MPTP administration. Mice were injected intraperitoneally (i.p.) with MPTP-HCl in 0.9% NaCl or saline (0.9% NaCl), using a chronic dosing regimen of 20 mg/kg every day for 14 days.

### Behavioral Tests

Rotarod test was performed 8 days after MPTP induction.

Rotarod performance was measured by an automated system (Med Associates Inc.). In brief, the animal was placed on an accelerating spindle (5-40 rpm) for 5 min per trial and three consecutive trials per day. A 20-min break was set in between each trial. The fall time from the spindle was auto-calculated by the system when the mouse fell off the spindle within the 5-min interval. The stay time was calculated by subtraction of the fall time from the 5 min, and the mean value of the stay time from three consecutive trials per day was used for statistical analysis.

### Immunofluorescence Staining of Mouse Brain

Mice were sacrificed to obtain the brain samples for immunostaining studies at the 8th day after MPTP induction. For tissue preparation, perfusion was performed on anaesthetized mice with PBS and then 4% PFA. Brains and spinal cords were post-fixed in 4% PFA overnight before embedding. All tissues were sectioned at 40 μm using a vibratome (Leica VT1000S Germany). To visualize TH (tyrosine hydroxylase)-positive dopaminergic neurons in substantia nigra, floating sections were incubated in PBS containing 5% bovine serum albumin (BSA) with primary and secondary antibodies. DAPI (1:1000, Beyotime C1002) were included in the secondary antibody incubation medium for nuclear staining, and then washed. After staining, the sections were mounted with Fluoromount-g (southrenbiotech 0100-01). Fluorescent images were collected by confocal microscopy (Nikon A1 Japan).

### Plasmids and Lentiviral Vectors

DNA fragments corresponding to full-length of TRIM72 were amplified from a mouse cDNA library by PCR and inserted into pCMV-N-3×Flag expression vector between Sall and Xhol sites using seamless Cloning kit (Beyotime) to generate Flag-tagged TRIM72. The series of Flag-tagged TRIM72 domain deletion: ΔRing domain (deletion of 14-69 domain); ΔB-box domain (deletion of 81-122aa); Δcoiled-coil domain (deletion of 135-232aa); ΔPRYSPRY domain (deletion of 278-470aa) construct was generated from the full-length of TRIM72 expression vector.

For TRIM72 stable expression cell line construction, DNA fragments of the series of Flag-tagged TRIM72 mutants or domain deletion or single domain were amplified from the corresponding TRIM72 expression vector by PCR and inserted into pLJM1-EGFP lentiviral vector between BsrGI and EcoRI sites to generate the series of Flag-tagged TRIM72 mutants or domain deletion or single domain fused with EGFP in lentiviral vector.

Lentivirus expressing Flag-tagged TRIM72 construct was generated from the corresponding EGFP fused with Flag-tagged TRIM72 vector.

Cell culture, Lentiviral Packaging and Lentiviral Infection

HEK293FT cells were maintained in DMEM (Invitrogen) with 10% fetal bovine serum (Gemini) in cell incubator (37° C., 5% CO₂). For lentiviral packing, HEK293FT cells were seeded in growth medium for three 10-cm culture dish. When reaching around 90% confluence, cells were co-transfected with VSVG (10 uμg), pxPAX2 (15 μg) and pLJMI-EGFP lentiviral vector or pLentiCRISPRv2 (Addgene) or pLenticas9-Blast (Addgene) (20 μg) using PEI (Sigma) following manufacturer's instructions and changed medium with fresh growth medium 5-6 h after transfection. The medium was harvested 72 hours after transfection and centrifugated at 20,000 rpm, 4° C. for 2 hours. After centrifugation, the lentivirus was enriched in the pellet. The lentivirus was resuspended using 100 μl DPBS and stored in −80° C.

HEK293FT cells or Hela cells were infected with indicated lentivirus. After 3 days of infection, the infected cells were selected with 2 μg/ml puromycin or 10 μg/ml blasticidin according to the plasmid containing resistance for at least a week. The puromycin- or blasticidin-selected cells were applied for further analysis.

### Cell Viability Assay

Cell viability was assessed using CCK-8. Cells were seeded in a 96-well plate at a density of 1.5×10³cells per well for Arsenite treatment or 7×10³ cells per well for H₂O₂ treatment. Arsenite (Sigma) was added into each well at concentration of 0.125 mM, 0.25 mM or 0.5 mM, and washed the cells after 2 hours incubation at 37° C. For H₂O₂-treatment experiment, scAAV9 infection (estimated multiplicity of infection (MOI): 10,000 vg/cell) was done for 24 hours at 17 hours after cell seeding. Then, H₂O₂ was added into each well at concentration of 300 μM, and washed the cells after 1 hours incubation at 37° C. When the cell has been processed by Arsenite or H202. a total of 10 μL of CCK-8 solution (Yeasen) was added to each well. After another 2 hours of incubation at 37° C., the optical density (OD) value of each well was measured using a microplate reader with an excitation wavelength of 450 nm. The cell viability of 293 FT was calculated. The experiment was repeated at least three times to obtain the mean value.

### Western Blot Analysis

Total protein content in cells was extracted using RIPA lysis buffer (50 mM Tris-HCl, pH 8.0, 150 mM NaCl, 1 mM EDTA, 0.1% SDS, 1% TritonX-100, 0.5% Sodium deoxycholate) supplemented with phenymethanesulfonyl fluoride (PMSF) and proteinase inhibitor cocktail (Bimake) and the lysate was incubated for 30 minutes on ice. After 12000 rpm centrifugation for 10 minutes, the supernatant was extracted and was incubated at 95° C. for 10 minutes after mixing with SDS loading buffer. Next, the proteins were separated by 10% of sodium dodecyl sulfate-polyacrylamide gel electrophoresis (SDS-PAGE) and transferred onto a polyvinylidene fluoride (PVDF) membrane. Afterward, the membranes were blocked using 5% nonfat milk for 1 hours at room temperature. Then incubated with diluted primary antibodies overnight at 4° C., including GAPDH (Ameribio) TUBULIN (Ameribio), TRIM72 antibody (a kindly gift from Dr. Jianjie Ma), TSG101 (Abcam), ITGAV (Abcam), H3(Abcam). And then the membranes were incubated with HRP-conjugated secondary antibodies at room temperature for 1 hour. Images were analyzed using the Fiji ImageJ to obtain the integrated intensities.

### Exosome Purification

The protocol for purifying exosomes from 100 ml supernatant of 293 FT cells included two steps of ultrafiltration and polyethylene glycol (PEG) precipitation. First, pour the collected supernatant into a centrifuge tube and centrifuge at 3000 xg for 20 minutes to remove cell debris. Then, filter the supernatant once with a 0.45 μm filter. After that, take a new Amicon ULTRA-15 ultrafiltration tube washed with PBS or autoclaved water. Then immediately add the supernatant to the ultrafiltration tube, and centrifuge at 3000 xg for 5-10 minutes. Discard the filtrate, continue to add the supernatant, and centrifuge at 3000 xg for 5-10 minutes until all the supernatant is introduced into the ultrafiltration tube. Then transfer the concentrate to a 50 mL centrifuge tube. Add isolation reagent to the concentrate. Mix samples thoroughly by vortexing or pipetting. Finally, leave the samples at 2-8° C. overnight. The next day, samples were centrifuged at 10,000 xg for 1 hour at 4° C. Discard the supernatant, the exosomes are in the pellet.

### Example 1

### AAV-TRIM72 Packaging

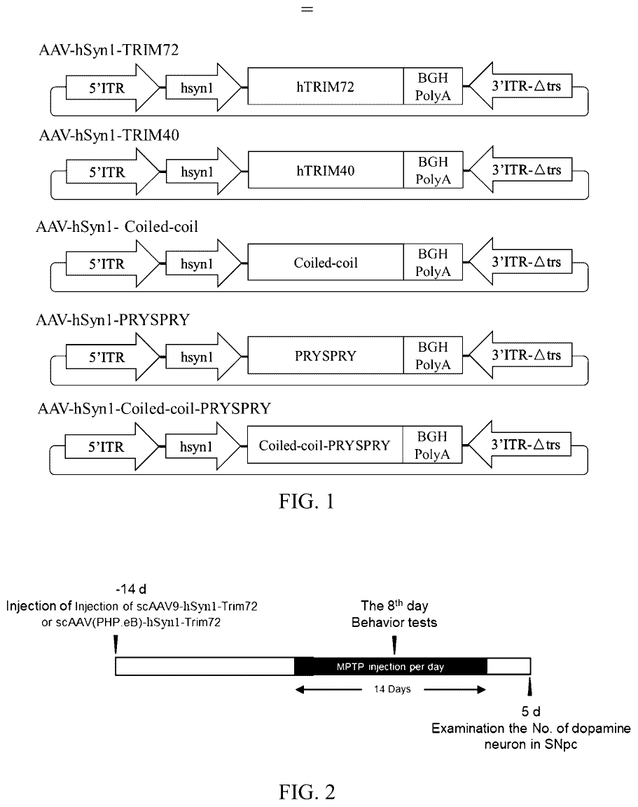

AAV packaging system is a commonly used triple-plasmid system. By simultaneously transfecting the three plasmids into mammalian cells (e.g. HEK293), all components required for AAV packaging can be expressed and assembled into virus particles in this cell. Here, we used a modified triple-plasmid system from PackGene (Guangzhou PackGene Biotech Co.,Ltd). This system consists of three plasmids: pAAV-ITR containing target gene, serotype vector pRepCapX and helper vector pADHelper. The target vector pAAV-ITR contains eukaryotic promoters and other components required for high levels of gene expression in mammalian cells when foreign sequences are cloned into polyclonal sites (MCS). The vector also contains AAV reverse terminal repeat sequences (ITRs) that guide virus replication and packaging. Vector pRepCapX contains AAV rep and CAP genes that encode replication proteins and viral capsid proteins. Stabilization of rep and CAP gene expression levels is a key step in obtaining desired high titer viral products. Vector pADHelper contains a collection of adenovirus genes VA, E2A, and E4 that are essential for cell production of high-titer viruses. FIG. 1 showed the construction of pAAV-ITR vectors for scAAVs.

### Example 2

scAAV-TRIM72 Alleviates Motor Dysfunctions and Dopaminergic Neuronal Loss in MPTP-Treated Mice

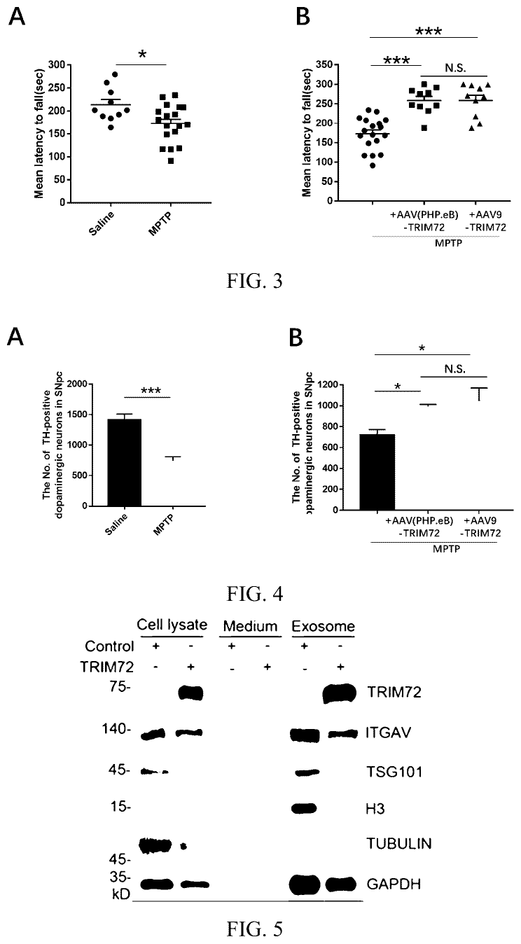

Here a MPTP-induced PD model was used to access the efficacy of scAAV-hSyn1-TRIM72 according to the administration procedure as shown in FIG. 2. MPTP exhibited a significant behavior defects and dopamine neuron loss, which were determined by rotarod performance and TH (tyrosine hydroxylase) immunostaining respectively (FIG. 3A and 4A). Using the well-established MPTP-induced PD model, AAV (PHP.eB) vectors at a dose of 10¹¹ vg/mouse or AAV9 vectors at a dose of 8×10¹² vg/mouse were injected into mice through retro-orbital intravenous injection at 14 days before the MPTP administration. Mice were equally divided into 3 groups. (1) MPTP group; (2) MPTP-scAAV (PHP.eB)-TRIM72 group; (3) MPTP-scAAV9-TRIM72 group. Firstly, we examined their motor skills on the Rotarod. MPTP-group mice showed an impaired rotarod performance, while MPTP-scAAV (PHP.eB)-TRIM72 mice or MPTP-scAAV9-TRIM72 mice exhibited significantly alleviated abnormalities in motor functions of rotarod behavior, suggesting scAAV-TRIM72 rescued motor functions in MPTP-treatment mice (FIG. 3B).

Furthermore, scAAV (PHP.eB)-TRIM72 or scAAV9-TRIM72 injection efficiently prevented these motor neuron loss (MPTP v.s MPTP-scAAV (PHP.eB)-TRIM72 mice or MPTP v.s MPTP-scAAV9-TRIM72 mice), which means that TRIM72 has a protective effect on motor neuron damage. (FIG. 4B).

### Example 3

### TRIM72 can be Secreted Through Exosome

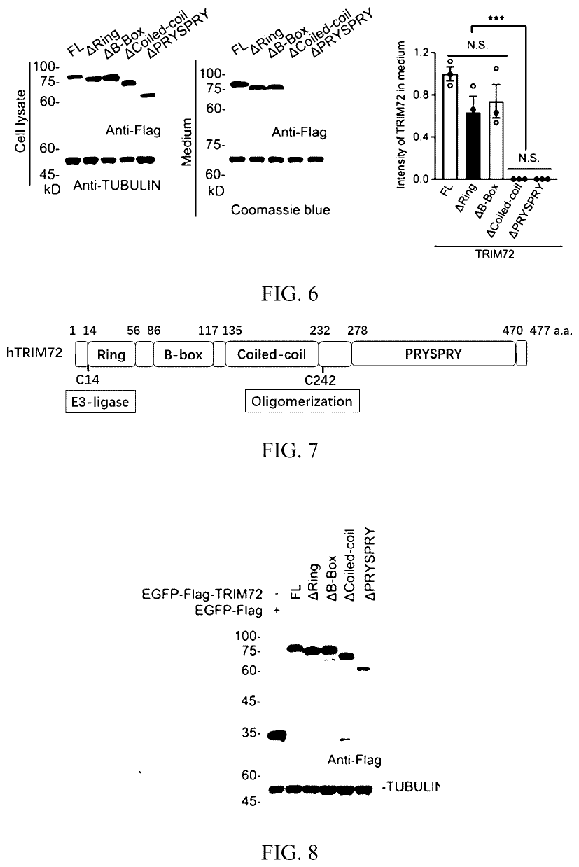

Exosomes are small extracellular biological vesicles released into surrounding body fluids through fusion of multivesicular bodies and the plasma membrane, which contain proteins, nucleic acids, lipids and other bioactive substances. Exosomes play an important role in the exchange of information between cells by releasing bioactive substances that fuse with receptor cell membranes or bind to cell surface receptors. Full-length TRIM72 or different domain-disrupted TRIM72 mutants were constructed and stably overexpressed in 293 FT cell line by lentiviral infection. We found that TRIM72 is enriched in TSG101-labeled exosomes, which means that TRIM72 could affect the biological processes of surrounding cells through the exosomal secretion pathway (FIG. 5). Further analysis showed that Coiled-coil domain and PRYSPRY domain are necessary for their exosome secretion, and the deletion of either domain will abolish the secretion of TRIM72 through exosomes (FIG. 6). Comparatively, removing the ring domain or B-box domain only has a limited impact on the secretion efficiency of TRIM72 (FIG. 6).

### Example 4

### Protective Effects of Truncated TRIM72 in Culture Cells

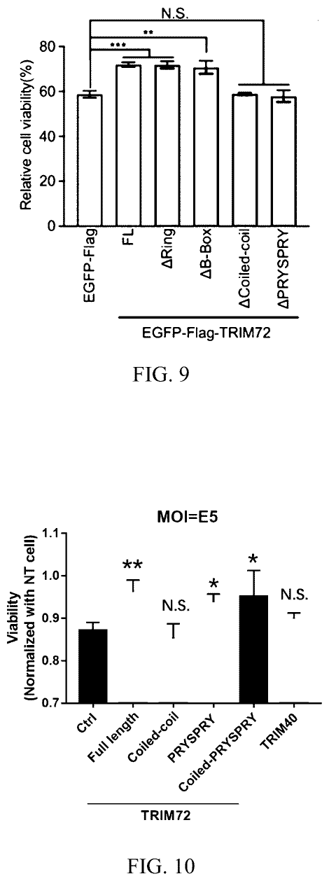

TRIM72 protein contains Ring finger motif, B-box domain, coiled-coil domain and PRYSPRY domain (FIG. 7). To investigate which domain of TRIM72 executes the protective function of anti-oxidative stress, full-length TRIM72 or truncated TRIM72 mutants were constructed and stably overexpressed in 293 FT cell line by lentiviral infection, followed by puromycin-selection (FIG. 8). CCK-8 was used to determine the cell viability after Arsenite treatment. The results demonstrated that coiled-coil domain- and PRYSPRY domain-deleted TRIM72 mutants abolished the protective effect of TRIM72, while Ring domain- or B-box domain-deleted TRIM72 mutants reserved similar protective effect as wildtype (FIG. 9).

To further investigate which key domains of TRIM72 protein are necessary to protect neurons, different domain-deleted TRIM72 were constructed and overexpressed in N2a cell line by scAAV9 infection (FIG. 1). CCK-8 was used to measure the cell viability after H₂O₂ treatment. Indeed, cell viability was decreased after H₂O₂ treatment (FIG. 10). Of note, overexpression of Coiled-PRYSPRY or the PRYSPRY single domain but not the coiled-coil single domain was sufficient to protect cells from oxidative stress, suggesting that either the Coiled-PRYSPRY construct or the PRYSPRY only could elicit protective effect (FIG. 10).

### Example 5

### TRIM72 but Not Other TRIM Proteins Protect Cells From Oxidative Stress

To investigate whether other TRIM proteins could also elicit protective effects from oxidative stress, TRIM40 were overexpressed in N2a cell line by scAAV9 infection (estimated multiplicity of infection (MOI): 10,000 vg/cell) (FIG. 1). CCK-8 was used to measure the cell viability after H₂O₂ treatment. Interestingly, treatment with TRIM72 rather than TRIM40 elicit protective effects from oxidative stress, which demonstrated that only TRIM72 but not other TRIMs could protect cells from oxidative stress (FIG. 10).

### Example 6

The critical Sites of TRIM72 Protein Which Protect Cells From Oxidative Stress

The series of Flag-tagged TRIM72 mutants: C14A (the cysteine at position 14 substituted by alanine); C242A (the cysteine at position 242 substituted by alanine) were generated from the wild-type TRIM72 construct by point mutation.

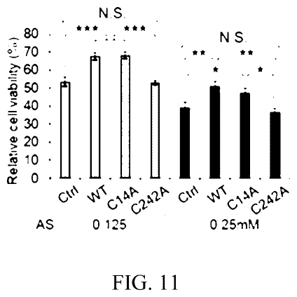

Previous study determined that TRIM72 senses changes in the oxidative environment and forms oligomer complex to complete membrane repair. A cystidine residue (C242) play a critical role in TRIM72 oligomer formation. In addition, the cystidine residue (C14) is critical for TRIM72 E3 ligase activity. Mutation of C242 into alanine (C242A) blocked TRIM72 protective effect, while its E3 ligase inactive mutant (C14A) reserved equal protective effect to wildtype (FIG. 11). It can be concluded that TRIM72 protects cells from oxidative stress and is mainly dependent on its oligomerization not E3 ligase activity.

While preferred embodiments of the present invention have been shown and described herein, it will be obvious to those skilled in the art that such embodiments are provided by way of example only. It is not intended that the invention be limited by the specific examples provided within the

1. A method for preventing and/or treating Parkinson's disease, comprising administering one or more TRIM72 protein modulators to a subject in need thereof.

2. The method of claim 1, wherein said TRIM72 protein modulator increases the expression and/or activity of said TRIM72 protein.

3. The method of claim 1, wherein said TRIM72 modulator is selected one or more for the group consisting of: a protein, a peptide, a peptidomimetic, a chemical compound, an antibody, a ribozyme, a small molecule chemical compound, a nucleic acid, a vector, and an antisense nucleic acid.

4. The method of claim 1, wherein said TRIM72 protein modulator comprises a TRIM72 protein or its variant or functional fragment thereof.

5. (canceled)

6. The method of claim 4, wherein said TRIM72 protein or its variant or functional fragment thereof comprises a full-length TRIM72 protein or a TRIM72 truncated protein or its functional fragment.

7-9. (Canceled)

10. The method of claim 6, wherein said TRIM72 truncated protein at least comprises the PRYSPRY domain or its functional fragment of a TRIM72 protein.

11. (canceled)

12. The method of claim 11, wherein said PRYSPRY domain comprises the amino acid sequence as set forth in SEQ ID NO: 6.

13-24. (canceled)

25. The method of claim 4, wherein said TRIM72 protein or its variant or functional fragment thereof comprises the amino acid sequence as set forth in SEQ ID NO: 2, 6, 7, 8, 9 and or 11.

26. The method of claim 4, wherein said TRIM72 protein or its variant or functional fragment thereof comprises an amino acid mutation at position C14.

27. (canceled)

28. The method of claim 4, wherein said TRIM72 protein or its variant or functional fragment thereof does not comprise an amino acid mutation at position C242.

29. The method of claim 4, wherein said TRIM72 protein or its variant or functional fragment thereof is secreted through exosome.

30. The method of claim 1, wherein said TRIM72 protein modulator comprises a nucleic molecule encoding the TRIM72 protein or its variant or functional fragment thereof.

31. The method of claim 1, wherein said TRIM72 protein modulator comprises a vector comprising a gene encoding said TRIM72 protein or its variant or functional fragment thereof.

32. (canceled)

33. The method of claim 31, wherein said vector is a recombinant adeno-associated virus (rAAV) expression vector.

34. The method of claim 31, wherein said vector comprises a general promoter or a neuron-specific promoter.

35. (canceled)

36. (canceled)

37. The method of claim 34, wherein said neuron-specific promoter is selected one or more from the group consisting of: an excitatory neuron-specific promoter, a brain neocortical and hippocampal excitatory neuron-specific promoter, a short neuron-specific promoter, a Dopaminergic neuron-specific promoter, a Glutaminergic neuron-specific promoter, a GABAergic neuron-specific promoter, a Cholinergic neuron-specific promoter and a Serotoninergic neuron-specific promoter.

38. The method of claim 34, wherein said neuron-specific promoter is selected from a group selected from: human synapsin (hSyn), Calcium/calmodulin-dependent kinase IIa (CamKIIa), c-fos, methyl CpG-binding protein 2 (Mecp2), Neuron-specific enolase (NSE), somatostatin (SST), human vesicular GABA (Gamma-Aminobutyric Acid) transporter (hVGAT), choline acetyltransferase (ChAT), Serotonin transporter (SERT) and tyrosine hydroxylase (TH).

39. (canceled)

40. The method of claim 33, wherein the serotype of the AAV capsid is selected from AAV1, AAV2, AAV5, AAV6, AAV8, AAV9, AAVrh, AAVDJ, and AA Vhull.

41. The method of claim 31, wherein said TRIM72 protein modulator comprises a cell, wherein said cell comprises said vector.

42. The method of claim 1, wherein said TRIM72 protein modulator comprises a fusion protein, wherein said fusion protein comprises said TRIM72 protein or its variant or functional fragment thereof.

43-129. (canceled)
